(19) United States
(12) Patent Application Publication
(10) Pub. No.: US 2010/0217052 A1
(43) Pub. Date: Aug. 26, 2010

(54) Catalyst For The Selective Hydrogenation Of Acetylenic Hydrocarbons And Method For Producing Said Catalyst

(75) Inventors: Sybille Ungar, Munich (DE); Richard Fischer, Bad Aibling (DE); Andreas Trautwein, Bruckmuehl (DE); Steve Blankenship, Radcliff, KY (US); Jennifer Boyer, Prospect, KY (US); Michael Urbancic, Louisville, KY (US); Andrzej Rokicki, Mountain Lakes, NJ (US)

Correspondence Address: RATNERPRESTIA, P.O. BOX 980, VALLEY FORGE, PA, 19482, US

(73) Assignee: SUD-CHEMIE AG, Munich (DE)

(21) Appl. No.: 12/601,985

(22) PCT Filed: May 30, 2008
(86) PCT No.: PCT/EP08/04327

§ 371 Date: Apr. 8, 2010

(30) Foreign Application Priority Data
May 31, 2007 (DE) 10 2007 025 315.1

(51) Int. Cl. C07C 5/08; B01J 31/12

(52) U.S. Cl. 585/250; 502/154

(57) ABSTRACT

A method for producing a catalyst, in particular for the selective reduction of acetylenic compounds in hydrocarbon streams. An impregnation solution is provided, which contains a mixture of water and at least one water-miscible organic solvent as solvent in which at least one active metal compound and also preferably at least one promoter metal compound is dissolved. A support is provided, the support is impregnated with the impregnation solution, and the impregnated support is calcined. Palladium is preferably used as active metal and silver is preferably used as promoter metal. Also, a catalyst as is obtained by the method and also its preferred use for the selective hydrogenation of acetylenic compounds.

### CROSS REFERENCE TO RELATED APPLICATIONS

This is a National Phase application of PCT application number PCT/EP2008/004327, filed May 30, 2008, which claims priority benefit of German application number DE 10 2007 025 315.1, filed May 31, 2007, the content of such applications being incorporated herein by reference.

### FIELD OF THE INVENTION

The invention relates to a method for producing a catalyst, in particular for the selective reduction of acetylenic compounds in hydrocarbon streams, a catalyst for the selective reduction of acetylenic compounds in hydrocarbon streams which has been obtained by the method and also its use for the selective reduction of acetylenic compounds in hydrocarbon streams.

### BACKGROUND OF THE INVENTION

Ethylene and propylene are important monomers for the production of plastics such as polyethylene or polypropylene. Ethylene and propylene are obtained predominantly from petroleum or petroleum products by thermal or catalytic cracking of long-chain hydrocarbons. However, the ethylene or propylene obtained from the cracking product still contains small amounts of acetylenic compounds such as acetylene or propyne. Before it is used further, e.g. in the polymerization of ethylene to form polyethylene, these acetylenic compounds have to be removed. For the polymerization of ethylene, the acetylene concentration has to be reduced to less than 5 ppm. For this purpose, the acetylene can be selectively hydrogenated to ethylene. The catalyst and hydrogenation process have to meet demanding requirements for this. Firstly, the acetylene should be removed as completely as possible by conversion into ethylene. Secondly, the hydrogenation of ethylene to ethane has to be prevented. For this purpose, the hydrogenation is carried out within a temperature range which is bounded by the “clean-up temperature” and the “runaway temperature”. For the purposes of the present invention, a “clean-up temperature” is the temperature at and above which appreciable hydrogenation of acetylene to ethylene is observed. A “runaway temperature” is the temperature at which appreciable hydrogenation of ethylene to ethane commences. The temperatures can be determined by, for example, measuring the hydrogen consumption of a defined gas mixture containing acetylene and ethylene as a function of the temperature. As catalysts for the selective hydrogenation of acetylene to ethylene in hydrocarbon streams, use is made of palladium catalysts which may also contain promoters such as silver or alkali metals. The palladium and, if appropriate, the promoters, in particular silver, are applied in the form of a shell to an inert and heat-resistant support material. The catalyst is produced by applying suitable salts of palladium and a promoter, for example palladium nitrate and silver nitrate, in the form of aqueous solutions to a porous support. The impregnation can be carried out in separate steps using a solution of the palladium compound and a solution of the silver compound. However, it is also possible to apply palladium and silver to the support in a joint impregnation step. The impregnated support is then calcined and reduced in order to convert the catalyst into the activated form.

DE 31 19 850 describes a process for the selective hydrogenation of a diolefin having at least 4 carbon atoms in a hydrocarbon mixture. The hydrogenation is carried out by means of hydrogen over a catalyst which simultaneously contains palladium and silver. The weight ratio of silver to palladium is from 0.7:1 to 3:1. The catalyst is produced by coimpregnation of a support with an aqueous solution of palladium and silver salts. U.S. Pat. No. 5,648,576 describes a process for the selective gas-phase hydrogenation of acetylenic hydrocarbons (C₂-C₃) to the corresponding ethylenic hydrocarbons. The catalyst is produced by coimpregnation of the support with an aqueous solution of the appropriate metal salts.

EP 0 064 301 discloses a catalyst for the selective gas-phase hydrogenation of acetylene. The catalyst is produced by two-stage application of palladium and silver.

A further catalyst for the selective hydrogenation of acetylenic hydrocarbons having two or three carbon atoms to the corresponding ethylenic hydrocarbons in the gas phase is described in EP 0 780 155. In the examples, solutions of palladium nitrate and silver nitrate in a nitrogen-containing acid are used for impregnation of the support.

In the case of many of the catalysts used hitherto, a layer of oligomers and polymers is formed on the surface during operation. This results in a decrease in the conversion and the [lacuna] oligomers and polymers. This results in a decrease in the conversion and the selectivity of the catalytic hydrogenation. Furthermore, the temperature range between “clean-up temperature” and “runaway temperature” also shrinks. The undesirable hydrogenation of ethylene to ethane therefore takes place at lower temperatures. Although the impurities on the catalyst can be removed by burning out by means of an oxygen-containing stream of air at elevated temperature, production has to be interrupted for regeneration of the catalyst, which incurs high costs. Furthermore, the fluctuating concentrations of acetylene and ethane in the ethylene produced make further processing more difficult.

It is therefore, in a first aspect, an object of the invention to provide a method for producing a catalyst for the selective reduction of acetylenic compounds, in particular acetylene and propyne, in hydrocarbon streams, with the catalyst avoiding the disadvantages of the prior art and allowing continuous and uniform hydrogenation over a long period of time without frequent regeneration of the catalyst. The catalyst should have a very broad temperature window between “clean-up temperature” and “runaway temperature”, with the temperature window not changing significantly over the life of the catalyst.

In a first aspect, this object is achieved according to aspects of the invention by a method having the features of claim 1.

Advantageous embodiments are subject matter of the dependent claims.

According to aspects of the invention, the at least one active metal from group 8 of the Periodic Table, preferably palladium, and (if present) the at least one promoter metal from group 1B of the Periodic Table, preferably silver, is/are applied to a support by (co)precipitation. As solvent, use is made of a mixture of water and at least one further organic solvent in which at least one active metal compound of an element of group 8 of the Periodic Table of the Elements and (if present) at least one promoter metal compound of an element of group 1B of the Periodic Table of the Elements are dissolved. The combined use of water and at least one organic solvent makes it possible to produce catalysts in which the active metals are present in very finely divided form, with at least 90% of the active metal particles and/or the promoter metal particles having a size of less than 6 nm. The advantageous effects of the invention are apparent even in the absence of a promoter metal (e.g. silver), i.e. when only one or more active metals is/are used. However, according to a preferred embodiment of the invention, at least one active metal and at least one promoter metal are used. The particles of active material formed by the active metal and, if present, the promoter metal can be applied in a very thin shell to the support by means of the impregnation. In one aspect of the invention, it has surprisingly been found that the penetration depth can be varied via the water content of the impregnation solution. Thus, the penetration depth of the impregnation solution increases with increasing water content of the impregnation solution.

In a preferred embodiment of the invention, the particles of the active metal or the active material preferably have a very narrow particle size distribution. This is surprisingly aided by the method of the invention.

The active metal and the promoter metal are preferably present together in the form of an alloy in the predominant part, i.e. preferably more than 50%, of the particles of active material applied to the support, so that intimate contact between the catalytically active metal and the promoter metal is achieved. Due to the small particle size and the high concentration of active metals in a thin outer shell (see below), a very high activity combined with a very high selectivity is achieved. Furthermore, the catalyst displays a significantly reduced tendency to form by-products which are deposited in the form of polymers on the surface of the catalyst. As a result, the catalyst displays a significantly prolonged stability of its properties, so that the cycles between regeneration of the catalyst can be significantly lengthened.

The method of the invention for producing a catalyst, in particular for the selective reduction of acetylenic compounds in hydrocarbon streams, is carried out by:
providing an impregnation solution which contains a mixture of water and at least one water-miscible organic solvent as solvent in which at least one active metal compound of an element of group 8 of the Periodic Table of the Elements and preferably at least one promoter metal compound of an element of group 1B of the Periodic Table of the Elements is dissolved;
providing a support;
impregnating the support with the impregnation solution.

The impregnated support is preferably calcined. Furthermore, the catalyst is preferably reduced, with this being able to be carried out in a separate step, for example after calcination or only in the reactor itself, e.g. during “start-up” of the catalyst. Preference is given to reduction by means of hydrogen before start-up of the catalyst.

In carrying out the method of the invention, an impregnation solution is firstly prepared. A mixture of water and a water-miscible organic solvent is used as solvent for this purpose. The organic solvent should preferably be completely miscible with water, so that no multiphase system is formed. The organic solvent can be either a pure compound or a mixture of a plurality of organic solvents. In the interests of simplicity, preference is given to using only a single organic solvent. At least one active metal compound and at least one promoter metal compound are present in solution in the solvent mixture. The preparation of the impregnation solution can be carried out in any way. Thus, it is possible to dissolve the at least one active metal compound or the at least one promoter metal compound in water and dissolve the other compound in each case in the organic solvent and then combine the two solutions. However, it is also possible firstly to prepare a solvent mixture and then dissolve the at least one active metal compound and the at least one promoter metal compound in this. For the dissolution, the solvent can be at approximately room temperature. However, it is also possible to heat the solvent to accelerate the dissolution process. The organic solvent and the at least one active metal compound and the at least one promoter metal compound are preferably selected so that a very concentrated solution of the at least one active metal compound or promoter metal compound is obtained.

As the at least one active metal compound and the at least one promoter metal compound, preference is given to selecting a compound which can be converted into the corresponding oxide by heating in air. Suitable active metal or promoter metal compounds are, for example, the carbonates, hydrogencarbonates, nitrates, salts of organic acids such as acetates, oxalates or citrates or acetylacetonates. The anions of the active metal or promoter metal salts are preferably selected so that a very concentrated impregnation solution can be prepared. A silver compound suitable as promoter metal compound is, for example, silver nitrate. A palladium compound suitable as active metal compound is, for example, palladium acetate, palladium acetylacetonate, palladium citrate, palladium oxide or a mixture thereof.

In addition, a support is provided. According to a broad aspect of the invention, any solid supports can be used. Preference is given to using customary supports as are known for the production of catalysts for the selective hydrogenation of acetylenic compounds. According to a preferred embodiment, the support is a porous support or a support having channels. The support can also consist of a largely or completely non-porous material which has a (porous) coating which can be impregnated. For this reason, the term “support” as used in the context of the invention includes coatings and coated materials. Suitable supports are, for example, Al₂O₃, in particular α-Al₂O₃, clays, aluminium silicates, SiO₂, ZrO₂, TiO₂, SiC, ZnO or any mixtures thereof, with Al₂O₃ being particularly preferred. The support preferably has a specific surface area in the range from 1 to 60 m²/g, preferably from 3 to 35 m²/g. The pore volume of the support is preferably from 0.1 to 1.5 ml/g, particularly preferably from 0.2 to 1.0 ml/g. The average pore diameter of the support is preferably from 10 to 300 Å, particularly preferably from 30 to 200 Å. In the case of a coated support, the above values for specific surface area and porosity relate to the coating.

The support can have any form. The support is particularly preferably provided in the form of a shaped body or a coating (see above). The shape of the shaped body can in principle be selected freely. According to a preferred embodiment, a suitable shape is, for example, a tablet or a pellet. The material on which the coating is present comprises, in one possible embodiment, channels of any shape which have a cross section in the range from 0.01 to 15 mm² or, for example, ceramics fired to high density, for example in the form of rings. The support can, if appropriate, additionally contain a customary binder and also further additives such as pore formers. Here, a person skilled in the art can make recourse to his knowledge for producing such bodies.

The support is then impregnated with the impregnation solution. Techniques known to those skilled in the art can be used for this purpose. The support can be steeped in the impregnation solution. The “incipient wetness” method in which the at least one active metal compound and the at least one promoter metal compound are dissolved in a volume of solvent which corresponds approximately to the pore volume of the support is preferably employed here. The pore volume does not have to be fully utilized. It is also possible, for example, to utilize only from 80 to 90% of the pore volume of the support. However, the at least one active metal compound and the at least one promoter metal compound can also be dissolved in a volume of solvent which is greater than the pore volume of the support, with excess impregnation solution being drained off or the solvent being evaporated. However, it is also possible, for example, to spray the impregnation solution onto the support, with the support preferably being kept in motion during spraying. It is also possible firstly to impregnate the support with an alkaline solution, for example with an alkali metal hydroxide solution, e.g. NaOH, and then to apply the impregnation solution to the pretreated support on which the at least one active metal or promoter metal compound is then precipitated in the form of its hydroxide. The impregnation is preferably carried out so that both the active metal compound and the promoter metal compound are concentrated in a thin shell on the outside of the support. In a particularly preferred embodiment, the support, e.g. a tablet or a pellet, is kept in motion during spraying of the solution and at the same time dried by means of a stream of gas. In the case of a coating, the layer thickness is, according to a preferred embodiment of the invention, prescribed by the coating. The impregnation solution can preferably either be passed through the channels present or a differently structured coated support can be impregnated by spraying.

The impregnated support is preferably dried. Drying can be effected after impregnation or can preferably be carried out during impregnation. Drying during impregnation is preferred since particularly thin shells are then obtained. Drying can be carried out by customary methods, for example by drying the impregnated support in an oven. Drying is preferably carried out by drying the impregnated support in a stream of gas, with the impregnated support preferably being kept in motion. Air can be used as gas for drying. However, preference is given to using a stream of inert gas, for example a stream of nitrogen, so that premature oxidation of the at least one active metal compound or promoter metal compound is prevented and uniform application of the at least one active metal compound or promoter metal compound to the support is thus achieved. Drying is preferably carried out at room temperature so that no decomposition of the at least one active metal compound or promoter metal compound occurs at this stage. The temperature used for drying is preferably in the range from 15 to 120° C., particularly preferably in the range from 25 to 100° C.

The preferably dried, impregnated support is subsequently calcined in order to fix the at least one active metal compound or promoter metal compound on the support. Calcination is carried out in customary apparatuses, for example a furnace such as a rotary tube furnace. During calcination, temperatures of more than 200° C. are preferably set. However, the temperature selected is preferably not too high so that, for example, flowing-together of the reduced metal particles on the surface of the support is avoided. Calcination is preferably carried out in an oxygen-containing atmosphere, particularly preferably in the presence of air. However, it is also possible for the calcination to be carried out completely or partly under an inert gas atmosphere. For example, calcination can firstly be carried out under an inert gas atmosphere and subsequently in the presence of air. The time for which calcination is carried out is dependent on the amount of catalyst to be calcined and on the calcination temperature and can be determined by a person skilled in the art by means of appropriate trials. The calcination time is preferably in the range from 1 to 20 hours, particularly preferably from 2 to 10 hours.

As active metal compound, it is possible to use compounds of the elements of group 8 of the Periodic Table of the Elements, with preference being given to ruthenium, rhodium, palladium, osmium, iridium and platinum. Palladium is particularly preferred.

As promoter metal compounds, it is possible to use compounds of the elements of group 1B of the Periodic Table of the Elements, namely copper, silver and gold, with silver being particularly preferred. In a preferred embodiment, silver is partly or entirely replaced by gold.

In a preferred embodiment of the method of the invention, the impregnation solution is prepared by preparing at least one first solution by dissolving the promoter metal compound, preferably silver compound, in water, preparing a second solution by dissolving the active metal compound, preferably palladium compound, in an organic solvent and combining at least the first solution with the second solution. It has been found that in this way metal particles having a very small diameter are obtained after calcination and reduction.

As indicated above, the amount of water and the amount of organic solvent are preferably selected so that a very concentrated impregnation solution is obtained. However, it has been found that the activity of the catalyst can be favourably influenced when the proportion of the organic solvent is not too small. The layer thickness (penetration depth of the impregnation solution) can be adjusted via the water content. The more water present in the solution, the greater the layer thickness. In a further aspect, the invention therefore provides a method of setting the penetration depth of an impregnation solution into a support, wherein the impregnation solution contains an organic solvent as described herein and water and the penetration depth is influenced via the water content of the impregnation solution.

In a preferred embodiment, the ratio (v/v) of water to the at least one organic solvent in the impregnation solution is therefore selected between 9.95:0.05 and 0.05:9.95, preferably between 0.1:9.9 and 2:8, particularly preferably between 0.1:9.9 to 1:9.

In a further preferred embodiment, the proportion of water in the impregnation solution, based on the total weight of water and organic solvent, is between 0.05 and 10% by weight. The organic solvent can in principle be selected freely, with preference being given to solvents which can be removed completely from the support by drying and calcination. To obtain a sufficiently high concentration of the at least one active metal compound and at least one promoter metal compound in the impregnation solution, preference is given to using polar organic solvents which are particularly preferably completely miscible with water. Particular preference is given to using oxygen-containing solvents which preferably contain from 1 to 5, particularly preferably from 1 to 3, oxygen atoms. These solvents preferably do not contain any further heteroatoms in addition to oxygen and therefore comprise only carbon, hydrogen and oxygen.

The at least one organic solvent is particularly preferably selected from the group consisting of ketones, carboxylic acids, carboxylic esters, alcohols and ethers, with ketones and ethers being particularly preferred. A ketone suitable as organic solvent is, for example, acetone or ethyl methyl ketone. A suitable carboxylic acid is, for example, formic acid or acetic acid, and a suitable carboxylic ester is, for example, methyl acetate. As alcohols, it is possible to use either monohydric or polyhydric alcohols. Suitable monohydric alcohols are, for example, ethanol and butanol. Suitable polyhydric alcohols are, for example, glycol and glycerol and also polyethylene glycols and polypropylene glycols. Suitable ethers are, for example, diisopropyl ether and tetrahydrofuran, with cyclic ethers being preferred. Particular preference is given to acetone and tetrahydrofuran as organic solvent.

To simplify processing and to enable the organic solvent to be removed easily during drying, the at least one organic solvent preferably has a boiling point at atmospheric pressure of less than 150° C., particularly preferably less than 100° C., very particularly preferably less than 80° C. However, the organic solvent should not have an excessively high volatility at room temperature in order to aid handling. The at least one organic solvent preferably has a boiling point at atmospheric pressure of more than 50° C.

It has been found that the properties of the catalyst can be favourably influenced when calcination is carried out at a temperature which is not too high. The inventors assume that at lower temperatures the combustion of the organic solvent is incomplete and carbon-containing residues therefore remain on the catalyst and these partially poison the catalyst and thereby increase the selectivity of the catalyst. The temperature for the calcination is preferably below 400° C., more preferably below 350° C., particularly preferably in the range from 200 to 300° C. The impregnation solution preferably contains the at least one active metal compound, preferably at least one palladium compound, and the at least one promoter metal compound, preferably at least one silver compound, in a ratio which corresponds approximately to the ratio sought for the at least one active metal compound and the at least one promoter metal compound in the finished catalyst or is identical to this. The at least one promoter metal compound and the at least one active metal compound are preferably present in the impregnation solution in a molar ratio of promoter metal/active metal (Ag/Pd) in the range from 1:1 to 10:1, preferably from 1:1 to 7:1, particularly preferably from 1.5:1 to 6:1.

The concentration of the at least one active metal compound, preferably palladium compound, in the impregnation solution is preferably selected so that the amount of active metal compound, calculated as metal and based on the weight of the support or the coating, is between 0.001 and 1% by weight, preferably between 0.005 and 0.8, particularly preferably between 0.01 and 0.5% by weight.

The concentration of the at least one promoter metal compound, preferably silver compound, in the impregnation solution is preferably selected so that the amount of promoter metal compound, calculated as metal and based on the weight of the support (or the coating to be impregnated), is between 0.001 and 1% by weight, preferably 0.005 to 0.8, particularly preferably 0.01 to 0.5% by weight.

Apart from the active metal and the promoter metal, the catalyst can contain further metal compounds. Here, particular preference is given to compounds of the alkali metals and the alkaline earth metals. Preferred alkali metals are sodium and potassium. A preferred alkaline earth metal is magnesium. These further metals or metal compounds can be applied to the support simultaneously with the at least one active metal compound and the at least one promoter metal compound or in a separate step. Customary methods, for example impregnation methods, can be used for applying the further metals or metal compounds to the support. As metal compounds, it is appropriate to use compounds which can be converted into the oxides of the metals by calcination in air. Suitable compounds are, for example, nitrates, hydroxides, carbonates, acetates, acetylacetonates, oxalates or citrates of the metals. The amount of further metal compound, in particular alkali metal compound, is selected so that the catalyst contains from 0.05 to 0.2% by weight of the at least one further metal, calculated as metal oxide and based on the weight of the catalyst. The atomic ratio of the at least one further metal to active metal is preferably between 2:1 and 20:1, more preferably 4:1 to 15:1. However, in a preferred embodiment the catalyst contains no further metals apart from active metal and promoter metal.

The method of the invention leads to a catalyst for the selective hydrogenation of acetylenic compounds in hydrocarbon streams which tolerates a relatively broad temperature range within which the selectivity remains high, i.e. none of or only a small proportion of the ethylenic compounds is reduced, and allows long operating periods before regeneration of the catalyst is necessary to maintain the productivity of the plant in question.

The invention therefore also provides a catalyst for the selective hydrogenation of acetylenic compounds in hydrocarbon streams as can be obtained, for example, by the above-described method. The catalyst comprises a support and particles of an active material comprising at least the active metal and silver located on the support, with at least 90% of the particles of the active material having a diameter of less than 6 nm.

In a preferred embodiment, at least 75%, preferably at least 80%, particularly preferably at least 85%, very particularly preferably at least 90%, of the particles of the active material are formed by an alloy which contains both the active metal and the promoter metal.

The inventors assume that the high activity of the catalyst combined with high selectivity is particularly favoured by the specific distribution of the active components in the shell and the small size of the particles of the active material, resulting in a large catalytic surface area being available for diffusion-controlled reactions, which has an advantageous influence on the activity of the catalyst.

Apart from the active metal and the promoter metal, the catalyst can also contain further metals or metal compounds. Suitable metal compounds are, for example, alkali metal compounds such as sodium or potassium compounds. These compounds of the further metals are preferably present in the form of their oxides on the support.

The particle size and the particle size distribution of the active material can be determined, for example, by means of [lacuna] on which the number and size of the particles of the active material is determined and the corresponding values are evaluated statistically. At least 150 particles are evaluated with the aid of electron micrographs at an enlargement of 150 000. The particle diameter is taken as the longest dimension of the particles visible in the electron micrographs.

The particles of the active material of the catalyst preferably have an average particle diameter (unweighted arithmetic mean) of less than 5.5 nm, particularly preferably less than 4.5 nm. The proportion of particles which are formed by an alloy and contain both the active metal and the promoter metal can in the case of palladium as active metal and silver as promoter metal be determined by adsorption of carbon monoxide on the surface of the particles of the active material and evaluation of the intensity of the absorption bands. Carbon monoxide adsorbed on palladium displays characteristic bands which can be assigned to different types of coordination of the CO to the surface. Proceeding from the model of closest-packed spheres onto whose surface CO molecules are bound, the CO molecule can be bound to an individual palladium atom (top), bridge two palladium atoms (bridge) or bridge three palladium atoms (hollow). The carbon monoxide is preferentially adsorbed on three palladium atoms, i.e. located at the gaps in the packing of the palladium atoms. Only at a high degree of coverage are the energetically less favourable positions (top and bridge) occupied. If silver atoms are introduced into the palladium, fewer positions at which the CO can coordinate at the gaps between three palladium atoms are available, so that as the silver content increases the positions at which the CO is coordinated to only one palladium atom (top) become preferred. At a constant degree of coverage of the particles of the active material, the ratio of the intensity of the bands assigned to adsorption at a gap (hollow) or on a single palladium atom (top) thus changes. Conversely, it is thus possible to draw conclusions as to the degree of alloying from the ratio of the intensities. Furthermore, the wave number at which adsorption of a CO molecule on a single palladium atom can be observed changes as a function of the degree of alloying. In the case of pure palladium, the bands for adsorption of a CO molecule on a single palladium atom (top) are observed at 2070-2065 cm⁻¹. With increasing degree of alloying, a shift to wave numbers in the range 2055-2050 cm⁻¹ is observed.

In the catalyst of the invention, both the active metal and the promoter metal are preferably concentrated in a very thin shell. In a preferred embodiment, at least 90% by weight of the active metal are present in a shell of the support which has a layer thickness measured from the outer surface of the support (or the coating) of not more than 250 μm, preferably not more than 200 μm, particularly preferably 150 μm. In a further embodiment of the invention, the promoter metal(s) is distributed in the same way. The inventors assume that the high selectivity of the catalyst is achieved as a result of the very thin shell and the specific distribution of the active components, since the molecules which diffuse into the catalyst, for example acetylene or ethylene, can come into contact with the active material for only a very short time.

Within the shell formed by the active material, i.e. active metal and promoter metal, preferably palladium and silver, the active metal preferably has a very distinct concentration maximum on the outer surface zone of the shell. In other words, the highest concentration of the active metal(s) and preferably also the promoter metal(s) is, according to this particularly preferred embodiment of the invention, within 80 μm, preferably 60 μm, in particular 50 μm, of the surface (outer surface) of the support (or the coating). The highest concentration is particularly preferably directly at the surface of the support (or the coating) and decreases in the direction of the interior of the support (or the coating).

The above-described method enables both the active metal and the promoter metal to be concentrated within a very thin shell in the support material. In the catalyst of the invention, the active metal and the promoter metal in the volume of the support preferably form a joint concentration maximum at the outer surface zone of the support (see above).

The particle size distribution of the active material, in particular the active metal (e.g. palladium) and preferably also the promoter metal (e.g. silver), has, in a particularly preferred embodiment, a maximum having a width at half height of less than 4 nm. The width at half height can be determined by plotting the number of particles against their diameter so as to give a curve having a maximum in the particle size distribution. The width at half height then corresponds to the width of the peak of the maximum at 50% of its height, measured from the zero.

The distribution of the active metal and the promoter metal in the support can be determined by preparing a section of the catalyst, for example by appropriately grinding and polishing of the support. The spatial distribution of the active metal or the promoter metal can then be determined by means of WDX spectroscopy (wavelength-dispersive X-ray diffraction) under an electron microscope. Here, a measurement head which is sensitive to the active metal, preferably palladium or the promoter metal, preferably silver, is moved over the specimen so that the distribution of the metal over the area can be determined.

The electron microprobe is a combination of a scanning electron microscope (SEM) and an X-ray fluorescence spectrometer.

A finely focussed electron beam impinges on the specimen. As in the case of SEM, this beam can be used for imaging the specimen. The experimenter can thus produce an enlarged secondary electron image of the specimen and within it seek the place at which he wishes to measure (in addition, the Jeol probe also has a camera which provides a light-optical image having an enlargement of 500×). At this place, the elements present can be identified and their concentration by mass can be determined.

The identification of the elements and the determination of the concentration are carried out as follows:

The electron beam impinges on the specimen at the measurement position and penetrates into the material. The penetration depth is in the order of from 1 to 3 μm and can be altered by changing the excitation voltage of the electron beam (at a higher excitation voltage, the penetration depth is greater). The electrons which have entered the specimen interact with the atoms of the specimen. Here, the electrons are slowed down and a continuous slowing-down spectrum whose upper limit is determined by the excitation voltage of the electron beam is emitted. In addition, the following process occurs: an electron knocks an electron out of the electron shell of an atom. As a result, a hole is produced in a shell (in terms of the Bohr model) and this is immediately replaced by an electron from a higher shell. In the process, this electron emits an X-ray photon having an energy corresponding to the energy difference between the two shells. The emitted photon can be absorbed by an electron from the electron shell and this electron then leaves the shell as an “Auger electron” or can leave the electron shell and be emitted from the specimen. The totality of the X-ray photons produced and emitted from the specimen in this way forms the “characteristic X-ray spectrum” which consists of lines having discrete energies. Since the energy levels of the electrons are characteristic of each other, the energies of the characteristic lines make it possible to determine the elements which are present in the specimen. In addition, the concentrations in which the elements are present can be determined from the intensities of the lines.

To carry out this identification of the elements and the determination of their concentrations, the X-ray radiation emitted isotropically from the specimen has to be analysed. This was carried out by means of wavelength-dispersive analysis (WDX): a narrow beam is taken from the emitted X-ray radiation by means of an orifice so that it impinges on an analyser crystal of a spectrometer. Depending on the orientation of this crystal relative to the incident radiation, a fixed wavelength is reflected from the surface (Bragg condition) and the reflected beam is recorded by means of a detector (the gas flow counter, scintillation counter).

The catalyst preferably contains in an amount in the range of from 0.001 to 1% by weight, more preferably 0.01 to 0.8% by weight, of the active metal, in particular palladium, based on the weight of the catalyst or the coating.

In a preferred embodiment, the catalyst contains in an amount of 0.001 to 1% by weight, preferably 0.005 to 0.8% by weight, of the promoter metal, in particular silver, based on the weight of the catalyst or the coating.

The catalyst comprises a porous inorganic support which can be selected from among all customary support materials. The inorganic support material is preferably selected from the group consisting of aluminium silicates, SiO₂, Al₂O₃, zeolites, kieselguhr, TiO₂, ZrO₂, ZnO, SiC and mixtures thereof. In principle, not only the support materials mentioned here but also all chemically inert, abrasion-resistant and heat-resistant support materials are suitable.

Preference is given to using Al₂O₃, particularly preferably α-Al₂O₃, as inorganic support material.

The catalyst preferably has a specific surface area measured by the BET method of from 1 to 80 m²/g, preferably from 2 to 45 m²/g.

Furthermore, the catalyst preferably has, based on the palladium, a CO adsorption in the range from 1000 to 5000 μmol/g. A method of measuring the CO adsorption is described below.

The catalyst can in principle be provided in any form, with the form of a shaped body (or coating, see above) being preferred. All geometries known to those skilled in the art, for example spheres, cylinders, tablets, stars and the corresponding hollow bodies, are possible. In the case of coatings, suitable forms are, for example, all ceramic supports fired to high density or metallic supports having channels of any shape or shaped bodies fired to high density, e.g. rings.

The shaped body is preferably configured as a sphere or tablet since the layer of the active material can be formed very precisely in the case of these shapes. The size of the shaped body varies as a function of the respective process conditions and can easily be adapted by a person skilled in the art. The shaped bodies used can have a uniform shape or be present as mixtures of various geometries.

Preference is given to a coating since the maximum layer of the active material or the maximum penetration depth of the impregnation solution containing the active and promoter metals can be predetermined by this.

The dimensions of the shaped bodies are selected in the range suitable for such applications. Suitable shaped bodies are, for example, spheres having a diameter of from 1 to 20 mm, preferably from 2 to 15 mm, or tablets having a diameter and a height in the range from 1 to 20 mm, preferably from 2 to 15 mm. Apart from the at least one promoter metal from among the elements of group 1B of the Periodic Table of the Elements, in particular silver, the catalyst can also contain further promoters. The further promoter is preferably selected from the group consisting of the compounds of the alkali metals and alkaline earth metals.

The catalyst of the invention has a high activity and selectivity in the hydrogenation of acetylenic compounds in hydrocarbon streams. The invention therefore also provides, according to one aspect, for the use of the above-described catalyst for the selective hydrogenation of acetylenic hydrocarbons in hydrocarbon streams. However, other uses of the catalyst of the invention are also encompassed by the invention, in particular other selective hydrogenations, for example of dienes.

The catalyst of the invention is particularly suitable for the selective hydrogenation of alkynes and dienes having from 2 to 5 carbon atoms, in particular in mixtures of hydrocarbons as are obtained by cracking. The hydrogenation can be carried out in the gas phase or in a mixed gaseous and liquid phase. Such processes are known per se to those skilled in the art. The reaction parameters, for example the hydrocarbon throughput, the temperature and the pressure are selected in a manner analogous to known processes.

The catalyst is particularly suitable for the selective hydrogenation of acetylene in ethylene streams (C2) and of propyne in propylene streams (C3).

The hydrogen is appropriately used in an amount which is from 0.8 to 5 times, preferably from 0.95 to 2 times, the amount required for stoichiometric reaction.

The hydrogenation process can be carried out in a single stage or in a plurality of stages.

In the selective hydrogenation of acetylene in C2 streams to ethylene, it is possible to set, for example, a space velocity of the C2 stream based on the catalyst volume in the range from 500 to 10 000 m³/m³, a temperature of from 0 to 250° C. and a pressure of from 0.01 to 50 bar.

In the selective hydrogenation of propyne in C3 streams, comparable parameters to those used in the selective hydrogenation of acetylene are set when the selective hydrogenation is carried out as a gas-phase process. If the process is carried out using a mixed gas/liquid phase, the space velocity is appropriately from 1 to 50 m³/m³.

### BRIEF DESCRIPTION OF THE FIGURES

The invention is illustrated below with reference to the methods of examination used, examples and figures. These serve only for the purpose of illustration and do not restrict the invention in any way. The figures show:

FIG. 1: various IR spectra of samples of a catalyst having different Ag/Pd ratios on which CO has been adsorbed;

FIG. 2: a size distribution of particles of the active material for two catalysts according to aspects of the invention and a comparative catalyst;

FIG. 3: a wavelength-dispersive X-ray spectrum (WDX) of a catalyst according to aspects of the invention.

### PREFERRED EMBODIMENTS OF THE INVENTION

### 1. Methods of Examination

### 1.1. Determination of the Size Distribution of the Particles of The Active Material

The determination of the particle size distribution is carried out by means of transmission electron microscopy (TEM). The samples are firstly reduced. For this purpose, a sample of the catalyst in its oxidic form is heated at 80° C. under helium (100 ml/min) and dried for 30 minutes. The sample is then reduced at this temperature in a stream of hydrogen (10 ml/min) for one hour. The samples obtained in this way are transferred directly into the electron microscope. For this purpose, the samples are treated with ultrasound and detached particles are collected on a grid. 7 images are employed in each case for particle analysis. Depending on the contrast difference between the particles of the active material and the support material, the images are enhanced by means of commercial image processing software. This has no influence on the number and size of the particles. The number and size of the particles were counted/measured at intervals of 1 nm. At least 150 particles were measured at an enlargement of 150 000× (see above).

### 1.2. Electron Microprobe with Wavelength-Dispersive X-Ray Diffraction (WDX)

The catalyst was firstly embedded in resin and subsequently ground to the place at which the measurement is to be carried out. Silicon carbide discs having a mesh size in the range 100-4000 (4000 last) and isopropanol as lubricant are used for this purpose.

The JXA8900 microprobe from Jeol by means of which the measurements on the catalysts were carried out has 5 wavelength-dispersive spectrometers which each have 2 different analysers which can be changed under software control. This makes it possible to measure up to 5 X-ray lines simultaneously. The simultaneous measurement ensures that the X-ray lines actually come from the same place on the specimen.

In the measurement of the catalysts, it was possible to measure the lines Pd Lα1, Ag Lβ1, Al Kα, O Kα and C Kα simultaneously. The beam parameters for the measurement were:

Beam voltage 20 kV
Beam current 20 nA
Measurement time:
Peak position: 300 s
Background 150 s; measurements were in each case carried out at two background positions.

For other elements, the corresponding available lines are employed for the measurement.

### 1.3. CO Adsorption

To determine the CO adsorption, the sample is firstly oxidized at 400° C. in a mixture of 80% of N₂ and 20% of O₂ for 1 hour in a sample chamber to remove organic impurities. The sample is subsequently firstly flushed with pure N₂ for 30 minutes and then reduced in a stream of hydrogen (40 ml/min) for one hour at the same temperature. The sample prepared in this way is reacted with CO. For this purpose, 5 pulses (15 mbar) of CO are introduced into the sample chamber and after 15 minutes the chamber is flushed with hydrogen. The sample is kept under a hydrogen atmosphere at 400° C. for 30 minutes. The adsorbed CO reacts quantitatively with hydrogen to form methane. The amount of methane formed can be determined by means of an FID.

### 1.4. Determination of the Proportion of Pd/Ag Alloy

The determination of the proportion of Pd/Ag alloy is carried out by measurement of the type of bonding of CO to the catalyst surface. The sample preparation is carried out in a manner analogous to that in the measurement of the CO adsorption, but without reduction of the bound CO to methane. After introduction of the CO into the measurement chamber, the sample is cooled to room temperature over a period of 60 minutes. The CO-laden catalyst samples are subsequently measured in an IR spectrometer. The peaks observed in the IR transmission spectrum can be assigned to various bonding states of the CO molecule to the palladium layer. In the case of a pure palladium surface, the peak maximum for linear bonding (linear, “top” (l)) of the CO molecule is at 2065-2070 cm⁻¹, for bridge bonding (bridge(edge)_{b}(e)) it is at 1950-1965 cm⁻¹ and in the case of multiple bridging (hollow(h)) it is at about 1910 cm⁻¹. In the case of an alloy with silver, the peaks shift correspondingly. From the peak ratio of the “top” at the wavelength of the pure palladium sample and at the wavelength of the sample containing silver and palladium and also the area ratio l/((h+b(e)), it is then possible to determine the degree of alloying. The alloy formation can be estimated from the relative ratio of the respective peak areas l/((h+b(e)): the greater this ratio, the higher the proportion of alloyed metal particles.

In the case of an alloy with silver, there is a characteristic shift in the position of the peaks. The degree of alloying can then be determined from the peak ratio for linearly bound CO at the wavelength of the pure palladium sample and at the wavelength of the sample containing silver and palladium. For this purpose, the contribution of the respective peak to the total area of the “on top” peak is determined.

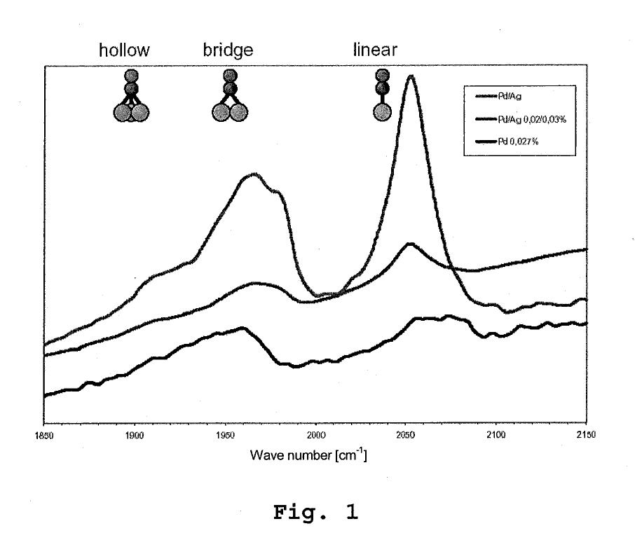

FIG. 1 shows by way of example an IR spectrum of catalyst samples having different Ag/Pd ratios on which CO has been adsorbed. It can clearly be seen that, compared to pure Pd catalysts, the proportion of linearly bound CO is increased for both bimetallic samples. This is particularly conspicuous in the case of the sample having the higher metal loading (blue curve). This can be attributed to the alloying with silver resulting in fewer hollow and bridge sites (3 or 2 continuous surface Pd atoms) being available for the adsorption of CO. The adsorption of CO on the bimetallic catalysts therefore occurs predominantly in a linear geometry on isolated surface Pd atoms.

### 1.5. Specific Surface Area (BET)

The determination is carried out by the BET method in accordance with DIN 66131; the BET method is also published in J. Am. Chem. Soc. 60, 309 (1938).

### 2. Examples

### 2.1. Production of Catalyst (A) According to Aspects of the Invention

3 ml of an 8.0% strength by weight aqueous silver nitrate solution are placed in a 0.5 l glass flask and admixed with 390 ml of a 0.069% strength by weight palladium acetate solution in acetone. The mixture is stirred at room temperature for 10 minutes. The solution obtained is applied by means of a ball coater to 500 g of aluminium oxide tablets having dimensions of 2×4 mm. The coated support bodies are dried at 80° C. under a stream of nitrogen for 1 hour and subsequently calcined at 300° C. in air for 3 hours. The catalyst A has a CO adsorption of 3600 μmol of CO/g of Pd.

### 2.2 Production of Catalyst (B) According to Aspects of the Invention

390 ml of a 0.069% strength by weight palladium acetate solution in acetone are admixed with 12 ml of distilled water at room temperature and stirred for 10 minutes. The solution is applied by means of a ball coater to 500 g of aluminium oxide tablets having dimensions of 2×4 mm. The coated support bodies are dried at 80° C. under a stream of nitrogen for 1 hour and subsequently calcined at 300° C. in air for 3 hours. Catalyst B has a CO adsorption of 7400 μmol of CO/g of Pd.

### 2.3 Production of Catalyst (C) According to Aspects of the Invention

4 ml of a 32.2% strength by weight aqueous silver nitrate solution are placed in a 0.5 l glass flask and admixed with 570 ml of a 0.08% strength by weight palladium acetate solution in acetone. The mixture is stirred at room temperature for 10 minutes. The solution obtained is applied by means of a ball coater to 500 g of aluminium oxide spheres having diameters of 2-4 mm. The coated support bodies are dried at 80° C. under a stream of nitrogen for 1 hour and subsequently calcined at 300° C. in air for 3 hours. The catalyst C has a CO adsorption of 2200 μmol of CO/g of Pd.

### 2.4 Production of Comparative Catalyst (D)

150 ml of a solution containing palladium nitrate (0.072% by weight) and silver nitrate (0.08% by weight) are applied to 250 g of aluminium oxide tablets having dimensions of 2×4 mm by means of a ball coater. The support bodies were subsequently dried and calcined as in Example 2.1, i.e. the coated support bodies were dried at 80° C. under a stream of nitrogen for one hour and subsequently calcined at 300° C. for 3 hours. The catalyst D has a CO adsorption of 700 μmol of CO/g of Pd.

### 2.5 Production of Comparative Catalyst (E)

150 ml of a solution containing palladium nitrate (0.072% by weight) are applied to 250 g of aluminium oxide tablets having dimensions of 2×4 mm by means of a ball coater. The coated support bodies are dried at 80° C. under a stream of nitrogen for one hour and subsequently calcined at 300° C. for 3 hours.

### 2.6 Production of Comparative Catalyst (F)

This example was carried out on the basis of Example 1 of EP 0 780 155. 150 ml of a nitric acid solution containing palladium nitrate (0.09% by weight) and silver nitrate (0.135% by weight) are sprayed onto 250 g of aluminium oxide tablets having dimensions of 2×4 mm. The coated support bodies are dried at 120° C. for 1 hour and subsequently calcined at 750° C. in air for 3 hours.

### 2.7 Comparison of the Particle Size Distribution

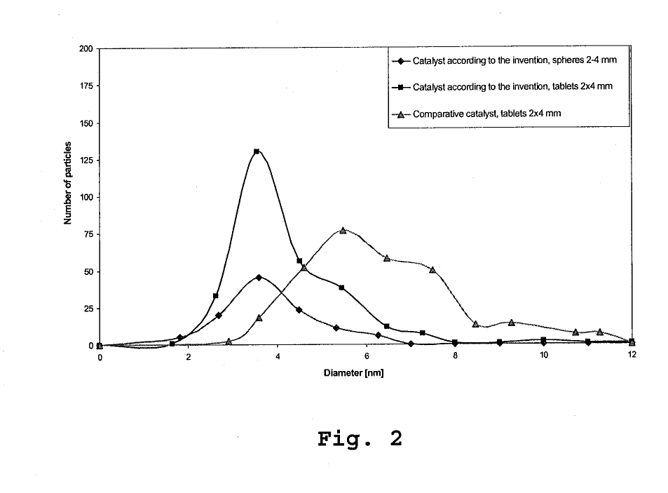

As can be seen from FIG. 2, the catalyst according to aspects of the invention both on the tablet-shaped (Example 2.1.) support material and on the spherical support material (Example 2.3.) has a narrow particle size distribution having a maximum at about 3.5 nm. The comparative catalyst D (Example 2.4.) has a very broad particle size distribution and only a local maximum at about 5.5 nm. The catalyst according to aspects of the invention is therefore present in a more precisely defined, narrow size distribution. This ensures constant properties when the catalyst according to aspects of the invention is used in the hydrogenation of acetylenic hydrocarbons. In addition, the narrow particle size distribution results in a broader temperature window (AT), higher selectivity and longer life compared to bimetallic catalysts containing Pd/Ag, for example catalysts produced as described in EP 0 780 155, Example 1.

### 2.8 Determination of the Operating Temperature Window and the Selectivity

25 ml of catalyst are introduced into a heated tube reactor and tested at GHSV of 7000 h⁻¹ and a pressure of 500 psig. The catalyst is firstly reduced in hydrogen at 94° C. for one hour, and the test is then started.

The crude gas composition is 1500 ppm of C₂H₂, 300 ppm of CO, 20% of H₂, 85 ppm of C₂H₆, 45% of C₂H₄ and CH₄ as balance. The temperature is increased until the clean-up temperature is reached. The clean-up temperature is the temperature at which a C₂H₂ concentration of <25 ppm is measured in the gas at the outlet.

The temperature is subsequently increased in 3° C. steps to the runaway temperature. The runaway temperature is defined as the temperature at which evolution of heat occurs and the hydrogen consumption is >4%.

The conversion is calculated as follows:

C₂H₂ conversion=(ppm of C₂H₂ at inlet−ppm of C₂H₂ at outlet)/(ppm of C₂H₂ at inlet)

The selectivity is calculated as follows:

C₂H₂ selectivity=(ppm of C₂H₂ at inlet−ppm of C₂H₂ at outlet−ppm of C₂H₆ at outlet+ppm of C₂H₆ at inlet)/(ppm of C₂H₂ at inlet)

TABLE 1
Comparison of the temperature window and the
selectivity in the hydrogenation of acetylene.

| | | | | Catalyst D | Catalyst E |
|---|---|---|---|---|---|
| | Catalyst A | Catalyst B | Catalyst C | (comparison) | (comparison) |
| Proportion of Pd | 0.02 | 0.02 | 0.03 | 0.02 | 0.02 |
| (% by weight) | | | | | |
| Proportion of Ag | 0.03 | — | 0.17 | 0.03 | — |
| (% by weight) | | | | | |
| Clean-up temperature | 53 | 51 | 50 | 49 | 48 |
| (° C.) | | | | | |
| Runaway temperature | 84 | 69 | 75 | 55 | 57 |
| (° C.) | | | | | |
| Selectivity (%) at the | 90 | 63 | 79 | 23 | −8 |
| clean-up temperature | | | | | |
| ΔT (° C.) | 31 | 18 | 15 | 6 | 9 |

### 2.8 Distribution of the Catalytically Active Elements in the Catalyst Particles

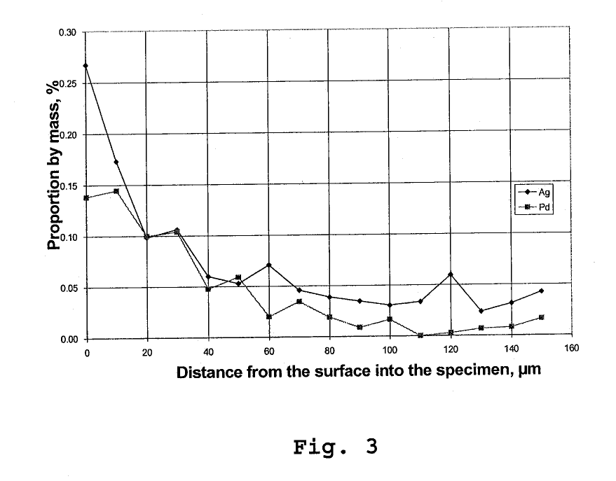

FIG. 3 shows the distribution of the catalytically active elements palladium and silver in the shell of the catalyst. Here, the elements silver and palladium are, as can be seen in the WDX spectrum, both present to a shell depth of 150 μm on the catalyst. The high enrichment of silver and palladium in the outer zone of the shell has a favourable effect on the performance of the catalyst.

1. A method for producing a catalyst, in particular for the selective reduction of acetylenic compounds in hydrocarbon streams, wherein:
an impregnation solution is provided which contains a mixture of water and at least one water-miscible organic solvent as solvent in which organic solvent at least one active metal compound selected from compounds of the elements of the group 8 of the Periodic Table of the Elements as well as preferably at least one promoter metal compound selected from compounds of the elements of group 1B of the Periodic Table of the Elements is dissolved;
a support is provided;
the support is impregnated with the impregnation solution;
the impregnated support is calcined.

2. The method according to claim 1, wherein at least one active metal compound and at least one promoter metal compound are used.

3. The method according to claim 2, wherein the impregnation solution is prepared by preparing at least one first solution by dissolving the at least one promoter metal compound in water, a second solution is prepared by dissolving the at least one active metal compound in an organic solvent and combining at least the first solution with the second solution wherein the impregnation solution is obtained.

4. The method according to claim 1, wherein the ratio (v/v) of water to the at least one organic solvent in the impregnation solution is selected between 9.95:0.05 and 0.05:9.95, preferably between 0.1:9.9 and 2:8, particularly preferably between 0.1:9.9 and 1:9.

5. The method according to claim 1, wherein the at least one organic solvent is an oxygen-containing solvent.

6. The method according to claim 1, wherein the at least one organic solvent is selected from the group consisting of ketones, esters, alcohols and ethers.

7. The method according to claim 1, wherein the at least one organic solvent has a boiling point at atmospheric pressure of less than 150° C.

8. The method according to claim 1, wherein the calcination of the impregnated support is carried out at a temperature of less than 400° C.

9. The method according to claim 1, wherein the at least one promoter metal compound and the at least one active metal compound are contained in the impregnation solution in a molar ratio in the range from 1:1 to 10:1.

10. The method according to claim 1, wherein the amount of active metal compound, calculated as metal and related to the weight of the support or the coating, is selected between 0.001 and 1% by weight.

11. The method according to claim 1, wherein the amount of promoter metal compound, calculated as metal and related to the weight of the support or the coating, is selected between 0.001 to 1% by weight.

12. The method according to claim 1, wherein the active metal compound is a palladium compound.

13. The method according to claim 1, wherein the promoter metal compound is a silver compound or a gold compound.

14. The method according to claim 12, wherein the at least one palladium compound is selected from the group of palladium acetate, palladium acetylacetonate, palladium citrate, palladium oxalate and mixtures thereof.

15. The method according to claim 1, wherein the support is dried during and/or after impregnation.

16. A catalyst for the selective hydrogenation of acetylenic compounds in hydrocarbon streams having a support and particles of an active material located on the support, the active material comprising at least one active metal selected from the elements of group 8 of the Periodic Table of the Elements and at least one promoter metal selected from the elements of group 1B of the Periodic Table of the Elements, wherein at least 90% of the particles of the active material having a diameter of less than 6 nm.

17. The catalyst according to claim 16, wherein the particles of the active material, in particular of the active metal and preferably also of the promoter metal, have an average particle diameter of less than 5.5 nm, in particular less than 4.5 nm.

18. The catalyst according to claim 16, wherein at least 90% of the active material is present in a shell of the support the shell having a layer thickness of at most 250 μm measured from the outer surface of the support.

19. The catalyst according to claim 16, wherein the particle size distribution of the active material, in particular of the active metal and/or of the promoter metal, has a maximum having a width half maximum of less than 4 nm.

20. The catalyst according to claim 16, wherein the maximum concentration of the active metal(s) and preferably also of the promoter metal(s) is located within 80 μm, preferably 60 μm, in particular 50 μm calculated from the surface (outer surface) of the support.

21. The catalyst according to claim 16, wherein the catalyst contains the active metal in an amount in the range from 0.001 to 1% by weight.

22. The catalyst according to claim 16, wherein the catalyst contains the promoter metal in an amount from 0.001 to 1% by weight.

23. The catalyst according to claim 16, wherein the catalyst has a specific surface area, measured according to BET from 1 to 80 m²/g.

24. The catalyst according to claim 16, wherein the active metal is palladium.

25. The catalyst according to claim 16, wherein the catalyst has a CO adsorption related to palladium of at least 1000 μmol/g, preferably in the range from 1000 to 10 000 μmol/g.

26. A method for the selective hydrogenation of acetylenic compounds, comprising contacting a hydrocarbon stream comprising an acetylenic compound with the catalyst according to claim 16.
